(19) United States
(12) Patent Application Publication
(10) Pub. No.: US 2025/0217759 A1
(43) Pub. Date: Jul. 3, 2025

(54) TRACKING SYSTEM FOR HOUSEHOLD ARTICLES AND SUPPLIES

(71) Applicant: Abhinav Tiwari, Piscataway, NJ (US)

(72) Inventor: Abhinav Tiwari, Piscataway, NJ (US)

(21) Appl. No.: 18/400,688

(22) Filed: Dec. 29, 2023

(51) Int. Cl. G06Q 10/087; G06Q 30/0601; G16H 10/60

(52) U.S. Cl. CPC G06Q 10/087; G06Q 30/0633; G16H 10/60

(57) ABSTRACT

A method of tracking household articles and supplies includes automatically maintaining an inventory of the household articles, including the most recent location, and an inventory of the supplies; comparing the inventory of the supplies to a predetermined re-order threshold; reporting the most recent location of one of the household articles upon request; and preparing a re-order list of the supplies when the inventory falls below the predetermined re-order threshold. A tracking system for household articles and supplies includes cameras mounted in each room of a real property; and a memory device electronically communicating with the cameras by way of a wireless network. The cameras have a view of an entire square footage in the room. The cameras continuously record image data collected and save the recorded image data to the memory device.

### BACKGROUND OF THE INVENTION

The present invention relates to tracking household articles and supplies and, more particularly, to a tracking system for household articles and supplies.

It is difficult to track consumption-based items, which can interfere with making the best use of health advice.

Individuals waste lots of time searching in the house or other property for day-to-day objects they forget after placing them somewhere in the house.

As can be seen, there is a need for a system that tracks the location of objects within a property and tracks the use of consumable items.

### SUMMARY OF THE INVENTION

In one aspect of the present invention, a method of tracking household articles and supplies comprises maintaining automatically an inventory of the household articles, including the most recent location; maintaining automatically an inventory of the supplies; comparing the inventory of the supplies to a predetermined re-order threshold; reporting the most recent location of one of the household articles upon receiving an inquiry from a user; and preparing a re-order list of the supplies when the inventory of any one or more of the supplies falls below the predetermined re-order threshold.

In another aspect of the present invention, tracking system for household articles and supplies comprises cameras mounted in each room of a real property, each camera having a sensor; a wireless network, electronically communicating with the cameras; a memory device electronically communicating with the cameras by way of the wireless network; wherein the cameras are configured to have a view of an entire square footage in the room; and wherein the cameras are operative to continuously record visual or image data collected and save the recorded image data to the memory device.

The system and method disclosed herein contribute significantly to improving the quality of life for Alzheimer's patients.

These and other features, aspects and advantages of the present invention will become better understood with reference to the following drawings, description, and claims.

### BRIEF DESCRIPTION OF THE DRAWINGS

FIG. 1 is a schematic view of an object location tracking system according to an embodiment of the present invention;

FIG. 2 is a flowchart of a method according to an embodiment of the present invention; and

FIG. 3 is a block diagram of a general and/or special purpose computer according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a tracking system, comprising cameras and a tracking device which tracks the last seen location of an item.

The system comprises cameras placed in the corners of rooms where they can be easily replaced and reprogrammed. The system includes a tracking device that tracks the exact location of objects in the house, without using a global positioning system (GPS), and identifies their last seen location. It tracks consumable goods by their usage, and it updates the consumer when it is necessary to refill a prescription or repurchase consumable goods. The system may purchase consumable goods on behalf of the consumer, comparing prices to obtain the best possible price. The system may evaluate the health of the user using a health record and the data collected by tracking consumption by the user.

Users can save lots of time searching for objects in the house after installing this tracking device. The system is particularly helpful for Alzheimer's patients who may have increased difficulty keeping track of items. The system generally performs actions on a processor faster than a human mind is capable of doing.

The processor may be programmed with tracking information and may be connected to the internet, where data regarding the location level information, including product details, may be stored. Data may be stored locally and/or on the cloud, with a biometric key. User identifiable information and medication information may be masked to protect the user's privacy. The processor extracts information from the collected image data, which may include, but is not limited to, who is present, the location of any object, medication adherence or compliance, daily routine, diet including what and how much is consumed, sleep cycle, and sleep hygiene. The processor may also track movement, both of individuals and of objects. The user may also manually input information. The processor retrieves medical information and correlates the extracted information with any of the user's medical conditions. The processor may operate on a back-end platform and utilize artificial intelligence (AI) in its extraction and correlation analysis. The processor produces a report containing feedback regarding the user's daily behavior and delivers it to the user's device, such as a mobile phone or personal computer.

In some embodiments, the processor may analyze articles brought into the house, identify any dangerous object, such as a gun or a knife, and immediately alert the user.

In some embodiments, the processor may track a quantity of a medication and/or a consumable and may notify the user when the quantity falls below a predetermined threshold. The processor may send an alert to the user, asking whether the system is to order replacement supplies and/or indicating where the user can obtain the replacement supplies.

In some embodiments, the processor may calculate the calorie content consumed by the user and may alert the user when a predetermined calorie threshold has been met. The alert may also suggest an action, such as exercise, prior to eating again.

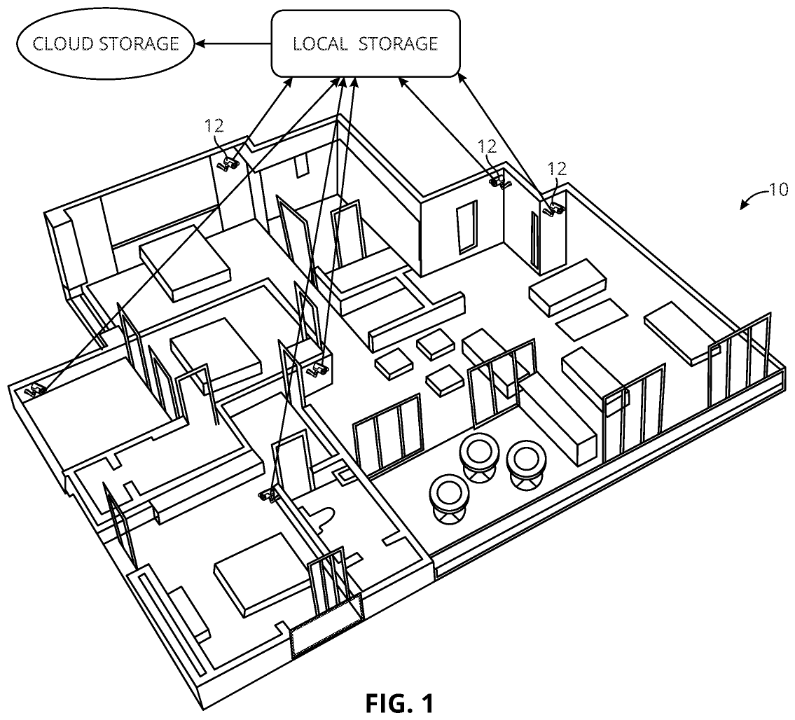

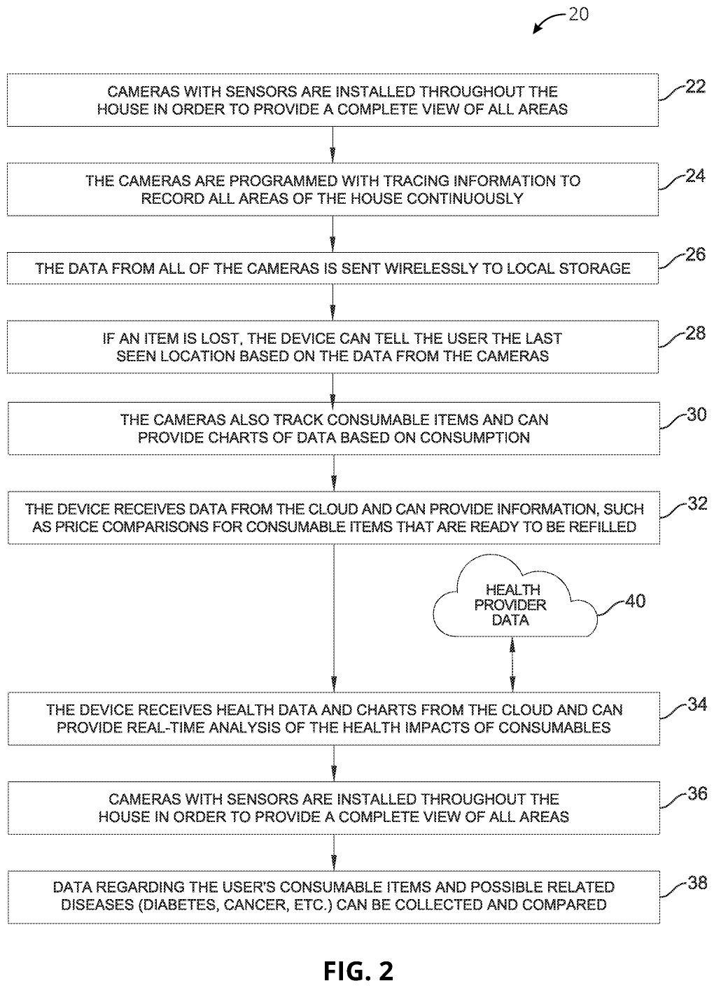

Referring to FIGS. 1 and 2, FIG. 1 illustrates placement of cameras 12 according to an embodiment of the present invention in a home 10. A flowchart of a process of using the system is shown in FIG. 2 and includes installing cameras 12 throughout the user's house, placed to provide a complete view of all areas 22. The cameras are programmed to record all areas of the house continuously 24. The data from all the cameras is sent wirelessly to local storage 26. If an item is lost, the device can tell the user the last-seen location based on the stored data from the cameras 28. The cameras also track consumable items and can provide charts of consumption data 30. The device receives data from the cloud and provides information such as price comparisons for tracked consumable items for which the supply is low 32. Using health provider data 40 stored in a cloud-based computer-readable memory, a processor provides real-time analysis of the health impacts of the consumable items in the house 34. If the user has diseases, such as diabetes or cancer, that may be related to the consumable items, the system collects data regarding their use and provides comparisons 38.

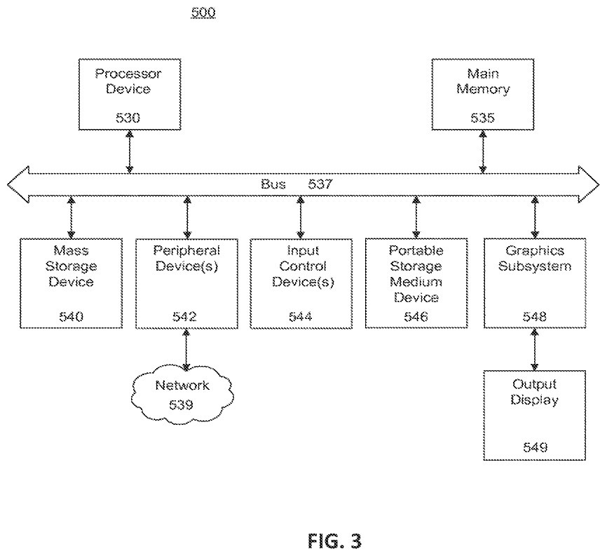

FIG. 3 is a block diagram of a general and/or special purpose computer 500, which may be a general and/or special purpose computing device, in accordance with some of the example embodiments of the invention. The computer 500 may be implemented as a conventional computer system, an embedded controller, a laptop, a server, a mobile device, a smartphone, a wearable computer, a set-top box, a kiosk, a vehicular information system, one more processor associated with a television, a customized machine, any other hardware platform, or any combination or multiplicity thereof. Computer 500 may be a distributed system configured to function using multiple computing machines interconnected via a data network or bus system.

Computer 500 may include without limitation a processor 530, a graphics processing unit (GPU), Application Specific Integrated Circuit (ASIC), or any combination thereof. Computer 500 may also include a main memory 535, and an interconnect bus 537. The processor 530 may include without limitation a single microprocessor or may include a plurality of microprocessors for configuring the computer 500 as a multi-processor system.

Processor 530 may be configured to execute code or instructions to perform the operations and functionality described herein, manage request flow and address mappings, and to perform calculations and generate commands. Processor 530 may be configured to monitor and control the operation of the components in computer 500. Processor 530 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof.

Processor 530 may be a single processing unit, multiple processing units, a single processing core, multiple processing cores, special purpose processing cores, co-processors, or any combination thereof. According to certain embodiments, processor 530 along with other components of computer 500 may be a virtualized computing machine executing within one or more other computing machines.

The main memory 535 stores, among other things, instructions and/or data for execution by the processor 530. The main memory 535 may include banks of dynamic random-access memory (DRAM), as well as cache memory.

The computer 500 may further include a mass storage device(s) 540, peripheral device(s) 542, non-transitory storage medium device(s) 546, input control device(s) 544, a graphics subsystem 548, and/or a display 549. For explanatory purposes, all components in computer 500 are shown in FIG. 5 as being coupled through bus 537. However, computer 500 is not so limited. Devices and systems of the computer 500 may be coupled through one or more data transport means. For example, processor 530 and/or the main memory 535 may be coupled through a local microprocessor bus. Mass storage 540, peripheral device(s) 542, portable storage medium device(s) 546, and/or graphics subsystem 548 may be coupled via one or more input/output (I/O) buses.

Mass storage 540 may include a hard disk, a floppy disk, a compact disc read-only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof.

Mass storage 540 may store one or more operating systems, application programs and program modules, data, or any other information. Mass storage may be part of, or connected to, computer 500. Mass storage 540 may also be part of one or more other computing machines that are in communication with computer 500, such as servers, database servers, cloud storage, network attached storage, and so forth.

Portable storage medium device 546 operates in conjunction with a nonvolatile portable storage medium, such as, for example, a compact disc read only memory (CD-ROM), to input and output data and code to and from computer 500. In some embodiments, the software for storing information may be stored on a portable storage medium and may be inputted into computer 500 via portable storage medium device 546.

Peripheral device(s) 542 may include any type of computer support device, such as, for example, an input/output (I/O) interface configured to add additional functionality to computer 500. For example, peripheral device(s) 542 may include a network interface card for interfacing computer 500 with network 539.

Input control device(s) 544 provides a portion of the user interface for a user of computer 500. Input control device(s) 544 may include a keypad and/or a cursor control device. The keypad may be configured for inputting alphanumeric characters and/or other key information. The cursor control device may include, for example, a handheld controller or mouse, a trackball, a stylus, and/or cursor direction keys. To display textual and graphical information, computer 500 may include graphics subsystem 548 and output display 549. Output display 549 may include a cathode ray tube (CRT) display and/or a liquid crystal display (LCD). Graphics subsystem 548 receives textual and graphical information and processes the information for output-to-output display 549.

Computer 500 may operate in a networked environment using logical connections through network 539 to one or more other systems or computing machines across network 539. Network 539 may include wide area networks (WAN), local area networks (LAN), intranets, the Internet, wireless access networks, wired networks, mobile networks, telephone networks, optical networks, or combinations thereof.

Network 539 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

Each component of computer 500 may represent a broad category of a computer component of a general and/or special purpose computer. Components of computer 500 are not limited to the specific implementations provided here.

Software embodiments of the example embodiments presented herein may be provided as a computer program product, or software, that may include an article of manufacture on a machine-accessible or machine-readable medium having instructions. The instructions on the non-transitory machine-accessible machine-readable or computer-readable medium may be used to program a computer system or other electronic device. The machine- or computer-readable medium may include, but is not limited to, floppy diskettes, optical disks, CD-ROMs, and magneto-optical disks or other types of media/machine-readable medium suitable for storing or transmitting electronic instructions. The techniques described herein are not limited to any software configuration. They may find applicability in any computing or processing environment. The terms “computer-readable”, “machine-accessible medium” or “machine-readable medium” used herein shall include any medium that is configured for storing, encoding, or transmitting a sequence of instructions for execution by the machine and that causes the machine to perform any one of the methods described herein. Furthermore, it is common in the art to speak of software, in one form or another (e.g., program, procedure, process, application, module, unit, logic, and so on), as taking an action or causing a result. Such expressions are merely a shorthand way of stating that the execution of the software by a processing system causes the processor to perform an action to produce a result.

Portions of the example embodiments of the invention may be conveniently implemented by using a conventional general-purpose computer, a specialized digital computer and/or a microprocessor programmed according to the teachings of the present disclosure, as is apparent to those skilled in the computer art. Appropriate software coding (instructions) may readily be prepared by skilled programmers based on the teachings of the present disclosure.

Some embodiments may also be implemented by the preparation of application-specific integrated circuits, field programmable gate arrays, or by interconnecting an appropriate network of conventional component circuits.

Some embodiments include a computer program product. The computer program product may be a storage medium or media having instructions stored thereon or therein which can be used to control, or cause, a computer to perform any of the procedures of the example embodiments of the invention. The storage medium may include without limitation a floppy disk, a mini disk, an optical disc, a Blu-ray Disc, a DVD, a CD or CD-ROM, a micro-drive, a magneto-optical disk, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the computer-readable medium or media, some implementations include software for controlling both the hardware of the general and/or special computer or microprocessor, and for enabling the computer or microprocessor to interact with a human user or other mechanism utilizing the results of the example embodiments of the invention. Such software may include without limitation device drivers, operating systems, and user applications. Ultimately, such computer readable media further includes software for performing example aspects of the invention, as described above.

Included in the programming and/or software of the general and/or special purpose computer or microprocessor are software modules for implementing the procedures, methods, processes, algorithms, and models described above.

The above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. For instance, although the flowcharts depict a serial process, some of the steps/processes may be performed in parallel or out of sequence or combined into a single step/process. The scope of the claims is intended to broadly cover the disclosed embodiments and any such modification. It should be understood, of course, that the foregoing relates to exemplary embodiments of the invention and that modifications may be made without departing from the spirit and scope of the invention as set forth in the following claims.

What is claimed is:

1. A method of tracking household articles and supplies, comprising:
maintaining automatically an inventory of the household articles, including a most recent location;
maintaining automatically an inventory of the supplies;
comparing the inventory of the supplies to a predetermined re-order threshold;
reporting the most recent location of one of the household articles upon receiving an inquiry from a user; and
preparing a reorder list of the supplies when the inventory of any one or more of the supplies falls below the predetermined re-order threshold.

2. The method of claim 1, further comprising reporting the re-order list periodically to the user.

3. The method of claim 1, further comprising re-ordering the supplies on the re-order list automatically.

4. The method of claim 1, further comprising comparing prices of the supplies on the reorder list and identifying a lowest price source for each of the supplies on the reorder list.

5. The method of claim 1, further comprising obtaining medical records of the user to determine a health status; analyzing the inventory of the supplies to determine any effect on the health status; and analyzing a frequency of re-ordering the supplies.

6. A tracking system for household articles and supplies, comprising:
cameras mounted in each room of a real property, each camera operative to collect image data;
a wireless network, electronically communicating with the cameras;
a memory device electronically communicating with the cameras by way of the wireless network;
wherein the cameras are configured to have a view of an entire square footage in the room; and
wherein the cameras are operative to continuously record visual data collected and save the recorded visual data to the memory device.

7. The tracking system of claim 6, wherein the further comprises a processor operative to track items within the real property without use of a global positioning system.

8. The tracking system of claim 6, wherein the memory device is located at the real property.

9. The tracking system of claim 6, wherein the memory device is located remotely and accessed via an internet connection.

10. The tracking system of claim 6, further comprising a processor operative to analyze the recorded visual data.
